(19) United States
(12) Patent Application Publication
(10) Pub. No.: US 2005/0234403 A1
(43) Pub. Date: Oct. 20, 2005

(54) Fluid discharge device and fluid delivery system including same

(75) Inventor: Cary Joe Atkinson, Wasilla, AK (US)

Correspondence Address: MICHAEL TAVELLA, 2051 BRIGADIER DRIVE, ANCHORAGE, AK, 99507, US

(73) Assignee: Atkinson Technologies, Inc., Nashville, TN (US)

(21) Appl. No.: 11/062,682

(22) Filed: Feb. 22, 2005

Related U.S. Application Data
(60) Provisional application No. 60/546,168, filed on Feb. 23, 2004.

(51) Int. Cl. A61M 1/00

(52) U.S. Cl. 604/151

(57) ABSTRACT

A fluid discharge device for delivering fluid from a container, a fluid delivery system including a combination of the fluid discharge device coupled the container for discharging fluid therefrom, a system for a sustained delivery of predetermined flow rates of a medium, and a combination of the system for a sustained delivery with the fluid delivery system above. The fluid discharge device has a fluid intake portion and a fluid outlet portion. The fluid intake portion is adapted to be placed within the container when the fluid discharge device is coupled to the container, and defines either perforations at least at end regions thereof or channels along a length thereof for discharging fluid from the container. The fluid outlet portion is adapted to deliver fluid discharged from the fluid intake portion to a region outside of the fluid intake portion. The fluid outlet portion may extend from the fluid intake portion outside the container when the fluid discharge device is coupled to the container for delivering fluid discharged from the fluid intake portion to a region outside of the container. Alternatively, the fluid outlet portion may corresponds to an exit end of the fluid intake portion, and is adapted to be attached to an exit port in an interior region of the container for delivering fluid discharged from the fluid intake portion to a region outside of the container.

### FIELD OF THE INVENTION

The present invention relates to devices for discharging fluid from containers, to devices for delivering medium from a canister, and to fluid delivery systems including such devices and/or systems.

### BACKGROUND OF THE INVENTION

The discharge and delivery of fluid from containers, such as, for example, intravenous fluid delivery bags, fluid soap containers, fluid food containers, or fuel tanks, is typically effected using an exit port or exit tube disposed at a drainage or discharge end of such containers. Generally, the discharge end is provided at a location that allows the discharge of fluid from a container as a function of a vertical orientation of the container to allow a discharge of fluid therefrom based on gravity. The above typically entails the provision of an exit port at a lower end of the container.

A disadvantage of the above configuration is that it does not ensure a discharge of fluid from the container in a reliable manner when the container is subject to static and/or dynamic forces that tend to shift the fluid within the container such that the fluid cannot reliably reach the exit port. For example, an intravenous delivery bag being used while being transported, such as when a patient is being transported via helicopter, for example in a combat situation, could be subject to “static” forces other than gravitational forces, for example by either laying flat on a floor, or bearing the weight of another object thereon, or being inadvertently folded. The intravenous delivery bag could also be subject to “dynamic” forces brought about as a result of a movement of the bag, thus having its fluid contents move within the bag during transport. Under such circumstances, the fluid within the intravenous delivery bag may not reach the exit port as readily as when the static and/or dynamic forces are not present. Forces applied on the intravenous delivery systems, for example when a patient is being transported, sometimes tend to temporarily shut off the delivery of fluid to the patient.

When the container is another type of container, such as for example, when the container is a fuel tank on an airplane, the tank could be subject to static forces other than gravitational forces, such as when the airplane changes its spatial orientation. The tank could further be subject to dynamic forces than tend to move the fuel within the tank, thus possibly impeding a reliable discharge of the fuel of the tank. Other similar situations occur with other types of fluid containers, such as fluid soap containers, fluid food containers, etc., that may be subject to any static and/or dynamic forces that may shift the fluid either permanently or temporarily away from the exit port of the container.

Additionally, the art has to date failed to propose a system that would allow a regulated delivery of a medium from a container, despite a spatial orientation of the container, and especially in circumstances where shifting forces are present.

To date, no solutions have been proposed to circumvent the above disadvantages.

### SUMMARY OF THE INVENTION

The above disadvantages are overcome by embodiments of the present invention, in which a fluid discharge device for delivering fluid from a container is provided. The device comprises a fluid discharge device for delivering fluid from a container, and a fluid delivery system including a combination of the fluid discharge device coupled to the container for discharging fluid therefrom. The fluid discharge device has a fluid intake portion and a fluid outlet portion. The fluid intake portion may include one or more subparts. The fluid intake portion is adapted to be placed within the container when the fluid discharge device is coupled to the container, and defines either perforations at least at end regions thereof or channels along a length thereof for discharging fluid from the container. The fluid outlet portion is adapted to deliver fluid discharged from the fluid intake portion to a region outside of the fluid intake portion. The fluid outlet portion may extend from the fluid intake portion outside the container when the fluid discharge device is coupled to the container for delivering to a region outside of the container. Alternatively, the fluid outlet portion may corresponds to an exit end of the fluid intake portion, and is adapted to be attached to an exit port in an interior region of the container for delivering fluid to a region outside of the container.

According to embodiments of the invention, the fluid discharge device may have any suitable shape. For example the fluid discharge device may include a single fluid discharge tube, a branching network of fluid discharge tubes, or a star-shaped network of fluid discharge tubes, among others.

According to one embodiment of the invention, the perforations extend along an entire length of the fluid discharge device. The perforations may further be distributed along the fluid discharge device in a staggered manner. According to one embodiment, especially where the container is an intravenous delivery bag, the perforations are defined at least at lateral regions of the fluid discharge device. The perforations may further be cylindrical in shape.

According to a further embodiment, the fluid discharge device comprises a single channeled fluid guide. Alternatively, the fluid discharge device may define any geometric shape as would be readily recognized by one skilled in the art. For example, the fluid discharge device could comprise a branching network of channeled fluid guides, or a star-shaped network of channeled fluid guides. The fluid discharge device may, by way of example, exhibit a cross-sectional profile in a plane perpendicular to its longitudinal axis which is one of X-shaped, star-shaped, Y-shaped or H-shaped, other shapes being possible.

According to a further embodiment, the fluid discharge device is made of a flexible material. According to yet another embodiment, the fluid discharge device is made of plastic. Preferably, and especially when the container is an intravenous delivery bag, the fluid discharge device and intravenous delivery bag are both made of the same flexible plastic material or of similar flexible plastic materials. Another aspect of the present invention comprises a combination comprising a container and a fluid discharge device as described above, the combination in this way constituting a fluid delivery system, wherein the fluid discharge device is coupled to the container for delivering fluid therefrom.

According to one aspect of the invention, the combination above is an intravenous fluid storage and delivery system that comprises: a product storage envelope being generally rectangular and having a top, a bottom, a front wall, a back wall and two oppositely disposed sides; an outer pressure envelope, also being generally rectangular and having a top, a bottom, a front wall, a back wall, and two oppositely disposed sides, the outer pressure vessel being placed so as to completely surround and cover said product storage envelope and further such that the bottom and sides of the outer pressure envelope are in continuous sealing contact with the bottom and sides of the product storage envelope such that a common seam exists around the perimeter of the envelopes; a fluid sealably stored in said product storage envelope; an outlet in communication with said product storage envelope to enable extraction of said fluid from the product storage envelope, the outlet comprising the fluid discharge device; and an inlet in communication with the outer pressure envelope to permit entry of external fluid such as gaseous medium into the outer pressure envelope.

According to another aspect of the invention, the combination above is an intravenous fluid storage and delivery system that comprises: a product storage envelope being generally rectangular and having a top, a bottom, a front wall, a back wall and two oppositely disposed sides; an outer pressure envelope, also being generally rectangular and having a top, a bottom, a front wall, a back wall, and two oppositely disposed sides, said outer pressure vessel being placed so as to completely surround and cover said product storage envelope and further such that said bottom and sides of said outer pressure envelope are in continuous sealing contact with the bottom and sides of said product storage envelope such that a common seam exists around the perimeter of the envelope and further such that the top of said outer pressure envelope is attached to said top of said inner product storage envelope with non-continuous seams and with a spatial gap therebetween, said non-continuous seams permitting the flow of external fluid such as gas from said spatial gap into said outer pressure envelope; a fluid sealably stored in said product storage envelope; an outlet in communication with said product storage envelope to enable extraction of said fluid from said product storage envelope, said outlet comprising the fluid discharge device; an inlet in communication with said outer pressure envelope to permit entry of the external fluid into said outer pressure envelope through said spatial gap; a canister of gas removably connected to said inlet; and a means to control the flow of external fluid or gaseous medium into said inlet.

Advantageously, embodiments of the present invention further encompass a system for a sustained delivery of predetermined flow rates of a medium, such as a gaseous medium to the inlet means of the intravenous delivery system described above. According to one embodiment, the system for a sustained delivery includes an actuator adapted to be depressed for effecting a release of gaseous medium from the canister, and configured such that amounts of depression of the actuator are proportional to corresponding flow rates of gaseous medium being delivered from the canister; and a mechanism coupled to the actuator for effecting various degrees of depression of the actuator and for sustaining the actuator at each of the various degrees of depression for causing a proportional sustained delivery of predetermined flow rates of the gaseous medium from the canister.

Preferably, the mechanism includes a rigid cap disposed on the canister and defines a threaded bore therein; and a correspondingly threaded screw threaded into the threaded bore and coupled to the actuator such that various degrees of threading-in or of threading-out of the screw cause corresponding various degrees of depression of the actuator and sustain the actuator at each of the various degrees of depression.

According to aspects of the present invention, the system for a sustained delivery according to the present invention may be coupled in combination with a intravenous delivery system either including or excluding a fluid discharge device according to the present invention for allowing a sustained delivery of predetermined flow rates of a gaseous medium for effecting a corresponding sustained delivery of intravenous fluid from the system, the above regardless of a spatial orientation of the intravenous delivery system, and/or regardless of the presence of shifting forces.

### BRIEF DESCRIPTION OF THE DRAWINGS

In the drawings, where like elements are referred to by like reference numerals:

FIG. 1 is a schematic, side elevational view of a first embodiment of the present invention including a first embodiment of a fluid discharge device according to the present invention;

FIG. 2 is an exploded view of an alternate embodiment of the present invention shown in FIG. 1;

FIG. 3 is a schematic, side elevational view of another embodiment of the present invention including the embodiment of the fluid discharge device of FIG. 1;

FIG. 4 is a cross-sectional view along lines 4-4 in FIG. 3.

FIG. 5 is a view similar to FIG. 4, showing the product storage envelope as being partially drained;

FIG. 6 is a view similar to FIG. 5, showing the product storage envelope as being further drained;

FIG. 7 is a view similar to FIG. 4, showing the product storage envelope as being fully drained;

FIG. 8 is a view of a top seam of the embodiment of FIG. 3 showing the flow of pressurized gas;

FIGS. 9a-9b show different configurations for the embodiment of the fluid discharge device of FIG. 1 according to the present invention;

FIG. 10 shows a perspective view of an alternate embodiment of a fluid discharge device according to the present invention;

FIGS. 11a-11d show respective cross-sectional profiles of a fluid discharge device according to FIG. 10 according to alternative embodiments of the present invention;

FIGS. 12a and 12b show different configurations for the embodiment of the fluid discharge device shown in FIG. 10;

FIG. 13 is a view similar to FIG. 3, including the embodiment of the fluid discharge device of FIG. 10;

FIG. 14 is a view similar to FIG. 6, including the embodiment of the fluid discharge device of FIG. 10;

FIG. 15 is a perspective view of a canister of pressurized gas including a system for the sustained delivery of a predetermined flow rate of a gaseous medium according to an embodiment of the present invention; and

FIG. 16 is a partially cross-sectional view of the canister of FIG. 15

### DETAILED DESCRIPTION OF THE INVENTION

Embodiments of the present invention concern a fluid discharge device having a fluid intake portion, and a fluid outlet portion. The fluid intake portion is adapted to be disposed within a container of fluid, and is further either perforated at least at end regions thereof or channeled along a length thereof for discharging fluid. According to one embodiment of the present invention, the perforations are cylindrical, are provided along an entire length of the fluid intake portion, and/or are distributed uniformly along a surface of the fluid intake portion. The fluid outlet portion is adapted to deliver fluid discharged from the fluid intake portion to a region outside of the fluid intake portion. According to one embodiment, the fluid outlet portion is adapted to extend from the fluid intake portion outside of the container. Alternatively, the fluid outlet portion may correspond to an exit end of the fluid intake portion, and be adapted to be attached to an exit port in an interior region of the container.

Advantageously, the fluid discharge device according to embodiments of the present invention has a configuration that allows enhanced access to different regions within the container for discharging fluid therefrom. Embodiments of the present invention are particularly advantageous when used under circumstances where the container is subject to static and/or dynamic forces that may tend to shift the fluid within the container to locations not readily accessible by a conventional exit port on the container, such as, for example, a weight acting on the container, or forces present when the container is being used while in transport. In such situations, embodiments of the fluid delivery system of the present invention advantageously provide enhanced access to different regions within the intravenous delivery bag as fluid shifts within the bag by virtue of the presence of such forces.

Embodiments of the present invention further concern a combination of a fluid discharge device such as the one described above, coupled with a container of fluid, such as, for example, an intravenous delivery bag, a fluid soap container, a fluid food container, or a fuel tank, in order to deliver fluid therefrom.

If the container is part of an intravenous storage and delivery system, it preferably includes, according to the present invention, a product storage envelope covered on both sides by an outer pressure envelope. The seams of both the product storage envelope and the outer envelope are sealed together along their perimeters resulting in the outer envelope forming a double wall around the product storage envelope. A fluid discharge device according to embodiments of the present invention is coupled to the product storage envelope and the outer envelope such that the fluid intake portion of the fluid discharge device is disposed within the product storage envelope, and such that the fluid outlet portion of the fluid discharge device serves to discharge fluid from the fluid intake portion. As pressure is added to the outer pressure envelope, the inner product storage envelope will be compressed, draining fluid from within the product storage envelope through the fluid discharge device until the product storage envelope is evacuated. Because the fluid is being forced out of the product storage envelope through a pressure applied within the outer pressure envelope (as opposed to the fluid being forced out by virtue of the force of gravity only), the fluid will be forced out regardless of the spatial orientation of the intravenous delivery bag of the intravenous storage and delivery system. Thus, the intravenous delivery bag may be placed horizontally, or in any given orientation, and fluid would still be drained from within the product storage envelope as long as pressure is being applied to the region within the outer pressure envelope. The intravenous delivery system proper, exclusive of the fluid discharge device described herein, is described in further detail in both U.S. Pat. Nos. 5,505,708 and 5,656,033, the contents of which are incorporated herein by reference in their entireties.

Advantageously, when used in combination as part of the outlet means in the intravenous storage and delivery system comprising the product storage envelope and outer pressure envelope described above, embodiments of the fluid discharge device according to the present invention allow enhanced delivery of fluid from the product storage envelope at least by virtue of increasing the availability of fluid inlet holes at locations within the intravenous delivery bag for the discharge of fluid from the product storage envelope. The above advantage is especially desirable in circumstances where static and/or dynamic forces, which tend to shift the fluid within the intravenous delivery bag, (both types of forces hereinafter referred to as “shifting forces”) are present.

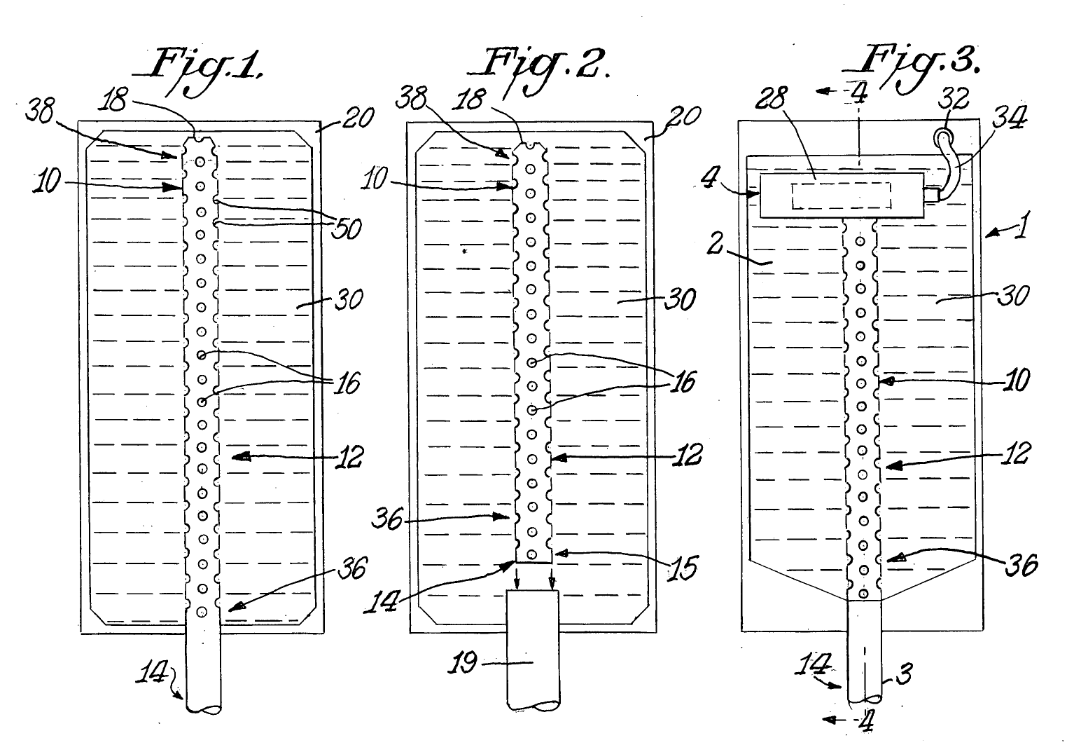

Referring now to the figures, FIGS. 1 and 2 show a schematic view of an embodiment of a fluid discharge device according to the present invention, where the fluid discharge device is a fluid discharge tube 10. As depicted in FIG. 1, fluid discharge tube 10 is shown in combination with a container 20. Tube 10 includes an elongated hollow body defining a fluid passageway therein. The hollow body has a fluid intake portion 12 and a fluid outlet portion 14. In the embodiment of FIG. 1, the fluid outlet portion 14 extends from fluid intake portion 12 outside of the container 20 for delivering fluid discharged from the perforations of the fluid intake portion. Alternatively, as shown in FIG. 2, the fluid outlet portion 14 may correspond to an exit end 15 of the fluid intake portion, and be adapted to be attached to an exit port 19 in an interior region of the container for forming, along with the exit port, a continuous fluid passageway from the interior of the container to the exterior of the container. In the embodiment of FIG. 2, the system is shown in exploded form, and the exit end 15 of the fluid discharge device is adapted to be inserted into the exit port 19 for forming the continuous passageway as suggested by the double arrows. It should be understood, however, that other configurations for attaching the fluid discharge device to an exit port are within the scope of the present invention, as readily recognizable by one skilled in the art.

Fluid intake portion 12 is adapted to be placed within container 20 and, in the shown embodiment, defines perforations 16 extending the entire length thereof for draining fluid 30 from the container through the perforations. However, according to embodiments of the present invention, the perforations are in any event provided at least at end regions thereof for providing enhanced access to an interior region of the container. The end regions correspond, respectively, to an end region defined on the fluid discharge device near the exit port, such as end region 36 shown in FIG. 1, and another end region defined at a tip of the fluid discharge device furthest from the exit port end region 36, such as end region 38 in FIGS. 1 and 2. When the container is an intravenous delivery bag according to embodiments of the present invention, the perforations provided on the fluid discharge device are provided at least at lateral regions of the same, that is, at regions thereof facing seams of the intravenous delivery bag. In FIG. 1, examples of lateral perforations are indicated with reference numeral 50.

According to embodiments of the present invention, the perforations may each be cylindrical, and/or may be distributed along a length on the fluid discharge device in a staggered configuration as shown in FIGS. 1-3. The above configuration of a fluid discharge device according to the present invention would ensure a reliable drainage of fluid from within a container of fluid than a convention exit port, in particular in circumstances such as those where shifting forces are present that would disturb a reliable discharge of fluid from within the container.

Embodiments of the present invention would include a fluid intake portion 12 which defines perforations only at predetermined locations along a length thereof, or only along a portion of a length thereof, and/or perforations shaped and/or distributed differently with respect to those shown in the embodiment of FIGS. 1 and 2. In addition, the container according to the present invention is not limited to an intravenous delivery bag, a fluid soap container, a fluid food container, or a fuel tank, but may include any container of fluid apt to be subject to shifting forces and designed for the drainage of fluid therefrom. For the purposes of the instant description, a “container of fluid” is either a container adapted to be filled with fluid, or a container already filled with fluid.

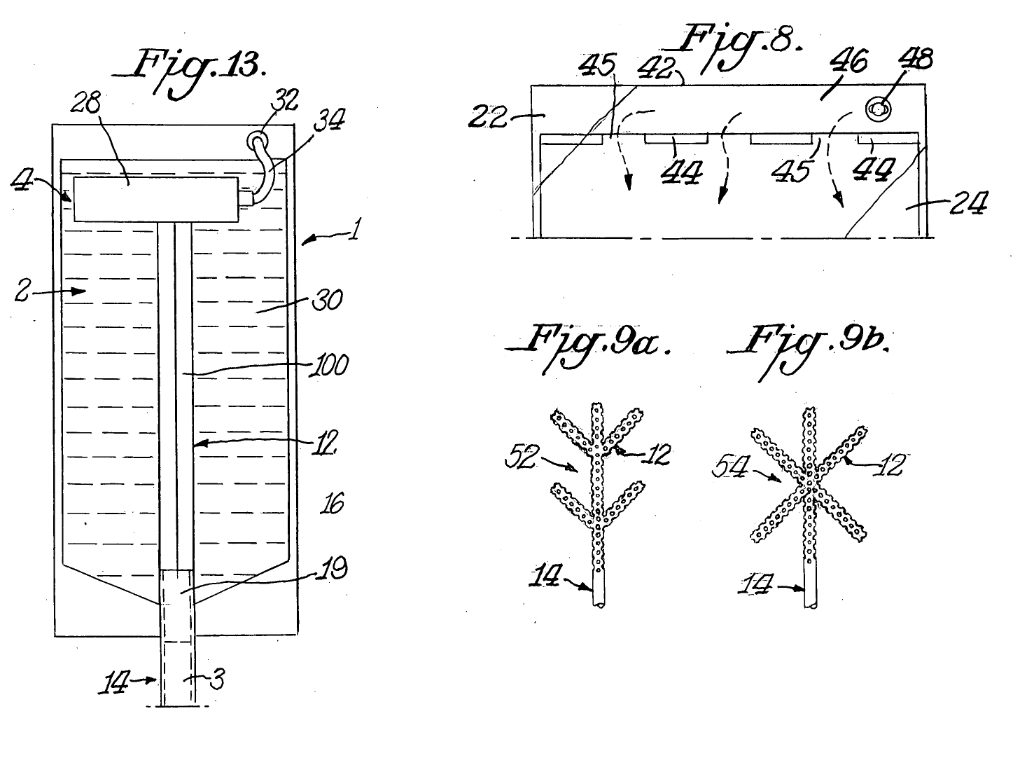

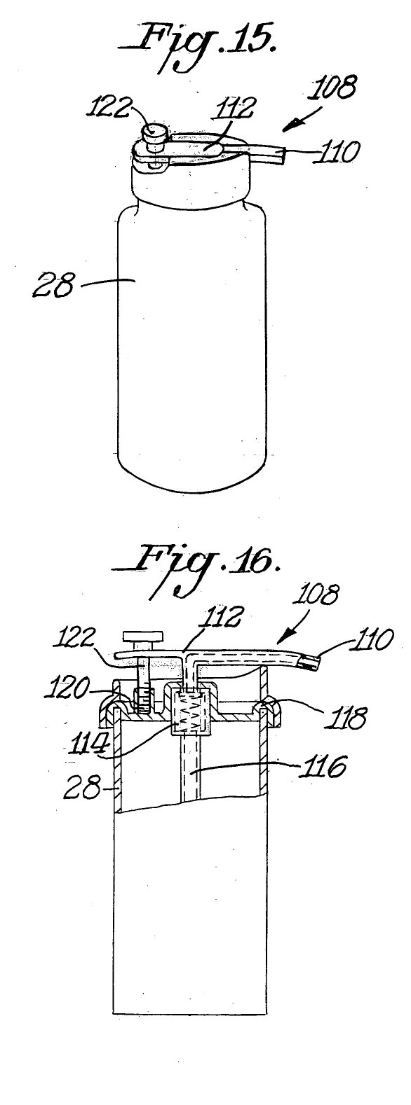

Another embodiment of a fluid discharge device according to the present invention is shown in FIGS. 10-14. According to the latter embodiment, the fluid discharge device is a channeled fluid guide 100 having intersecting walls, such as walls 102/104 shown in FIG. 10, which extend in a longitudinal direction of the channeled fluid guide in order to define similarly extending channels or grooves 106 at the intersection site thereof. The channels 106 are defined at a fluid intake portion 12 of the channeled fluid guide, and are effective for guiding fluid along a longitudinal direction of the channeled fluid guide for delivering fluid from regions along the channeled fluid guide 100 to the fluid outlet portion 14 as shown. The fluid intake portion 12 in the shown embodiment of the fluid discharge device thus corresponds to a portion of the channeled fluid guide 100 that is adapted to be disposed inside a container. In the embodiment of FIG. 10, the fluid outlet portion 14 is adapted to extend from fluid intake portion 12 outside of a container 20, such as the one shown in FIG. 2, for delivering fluid discharged from the channels of the fluid intake portion. As shown, the fluid outlet portion 14 may correspond to a tapering of the fluid intake portion 12, and is adapted to extend outside of a container by being for example inserted into an exit port of the container. Alternatively, as shown by way of example in FIG. 13, the fluid outlet portion 14 may correspond to an exit end 15 of the fluid intake portion and not be adapted to extend outside of a container, but rather be adapted to be attached to or inserted into an exit port 19 in an interior region of the container for forming, along with the exit port, a continuous fluid passageway from the interior of the container to the exterior of the container.

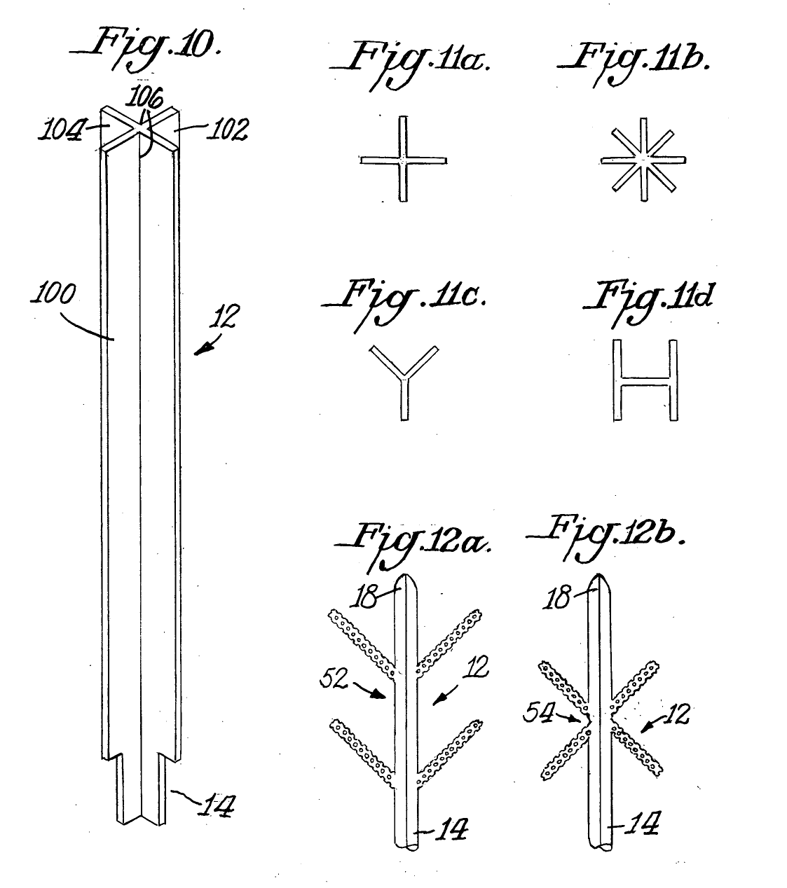

A channeled fluid guide 100 according to embodiments of the present invention may have any cross-sectional configuration where intersecting, longitudinally extending walls define respective channels or grooves therebetween, such as any of the cross-sectional configurations shown by way of example in FIGS. 11a-11d. The shown channeled fluid guide may be used instead of the fluid discharge tube 10 in any of the embodiments of the present invention apt to include the fluid discharge tube 10, such as any of the embodiments of the present invention depicted in FIGS. 1-7.

Where the combination according to embodiments of the present invention is an intravenous delivery system, such as the one shown in the embodiments of FIG. 3 or 13, the container and fluid discharge device may both be preferably made of a flexible material. More preferably, the container and fluid discharge device are both made of a flexible plastic, and, most preferably, they are both made of the same flexible plastic. Preferably, and especially where the container has thin walls made of a flexible material, such as in the case of conventional intravenous delivery bags, the end of the fluid intake portion 12 of fluid discharge tube 10 may be a domed end 18 as shown in FIGS. 1, 2, 4-7, 9a and 9b. In such a case, and in other embodiments of the fluid discharge tube and container of the present invention as would be within the knowledge of one skilled in the art, one or more perforations are additionally provided at the tip of end region 38 on the dome as shown in FIGS. 1, 4-7, 9a and 9b. In a similar situation, the end of the channeled fluid guide 100 may be a domed end 18 as shown by way of example in the embodiments of FIGS. 12a and 12b. A domed end may be provided on the fluid discharge device in order to prevent interference with the thin walls of the container.

Embodiments of the fluid discharge device according to the present invention may be used in combination with an intravenous delivery bag as part of an intravenous fluid storage and delivery system. Preferred embodiments of the system is shown in FIGS. 3 and 13, embodiments of the fluid discharge device configured as a fluid discharge tube 10 as seen in FIG. 3, or configured as a channeled fluid guide 100 as seen in FIG. 13. The system, exclusive of the fluid discharge device described herein, as noted above, has been described in further detail in both U.S. Pat. Nos. 5,505,708 and 5,656,033.

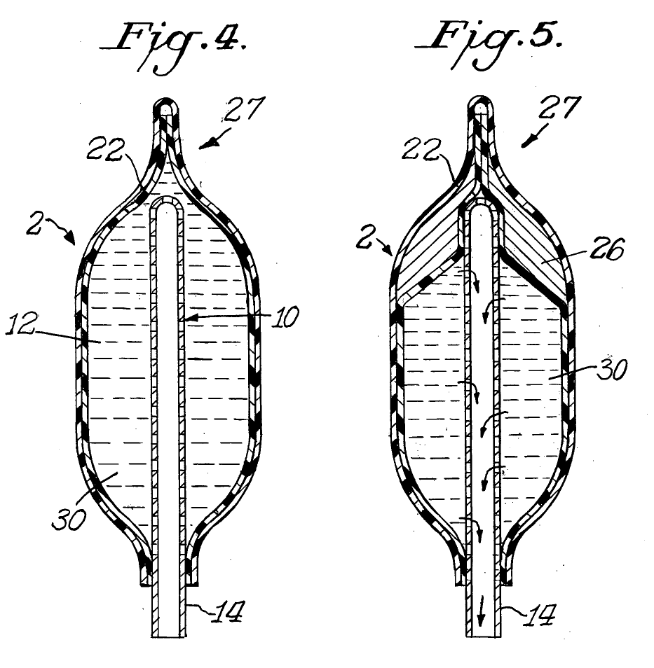

FIG. 4 is a cross-sectional view of the system of FIG. 3, and shows the system as including a fluid discharge tube 10. Alternatively, according to embodiments of the present invention, the fluid discharge tube 10 could be replaced with a channeled fluid guide 100 such as for example the one shown in FIG. 10.

In both FIGS. 3 and 13, the intravenous envelope system 1 shown has a fluid carrying assembly 2, an exit port 3, and an inflation system 4. A valve (not shown) in the exit port 3 prevents the escape of fluid from carrying assembly 2 until drainage of the fluid becomes necessary. The carrying assembly 2 in the shown embodiment corresponds to a container of fluid according to the present invention. The system is further equipped with a fluid discharge tube 10 (FIG. 3) or with a channeled fluid guide 100 (FIG. 13) for enhancing a delivery of fluid from assembly 2.

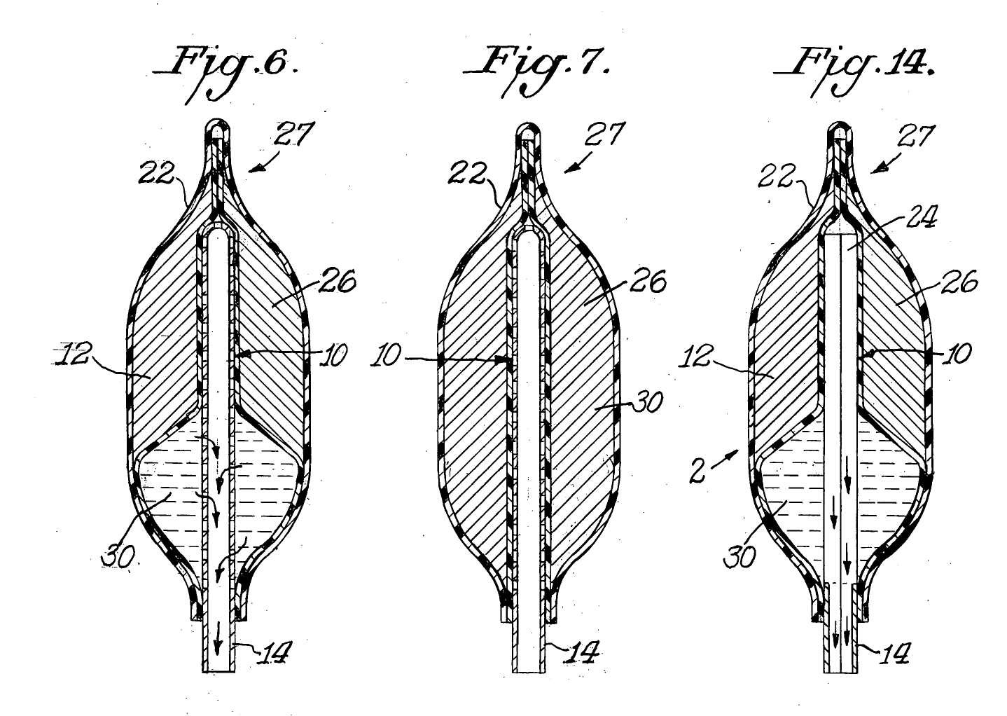

In the embodiment shown, and as better seen in FIGS. 4-7 and 14, the assembly 2 includes an outer pressure envelope 22 and an inner product storage envelope 24. The product storage envelope contains a fluid 30 therein. As shown in FIGS. 3-7, 13 and 14, the outer pressure envelope and inner pressure envelope are sealed together at their sides as shown, and at a top region thereof at seal 26 as shown in FIGS. 4-7 and 14. Seal 26, as described in further detail in the above-mentioned U.S. patents, includes tacked portions and as shown in FIG. 8, in order to allow pressurized gas to flow. As shown in FIG. 8, the folded top edge 40 of the product envelope 24 does not go completely to the top folded edge 42 of the outer envelope 22. This gap creates an air chamber to permit the entrance of pressurized gas to flow. The top seams 44 are tacked together as shown. This secures the outer envelope 22 to the product envelope 24. Use of tacking creates gaps 45 through which pressurized gas may flow from the upper chamber 46 into the outer pressure envelope 22. The seam structure is identical on both sides of the device. A pressure valve 48 is provided on the envelope as shown. This valve is similar to those found on many inflatable products such as air mattresses, beach balls, etc. As the gas is released into the canister, it flows into the valve 48 and proceeds to fill the outer pressure envelope 22. The system described above is designed to provide equal pressure to both sides of the outer pressure envelope.

The inflation system 4 for inflating a space between the outer pressure envelope 22 and the inner product envelope 24 may be identical or similar to the one described in the above-mentioned U.S. patents, and includes a canister 28 configured to deliver gaseous medium into the outer pressure envelope through gas passage 34 and inlet 32.

In the alternative, the inflation system 4 may, according to embodiments of the present invention, include a system that allows a sustained delivery of predetermined flow rates of a medium, such as a gaseous medium to gas passage 34 of FIGS. 3 and 13, or to gas passage 34 of an intravenous delivery system not including a fluid discharge device. An example of such a system for a sustained delivery according to embodiments of the present invention is shown schematically in FIGS. 15 and 16. Here, a canister of 28 includes a delivery cap 108 defining a nozzle 110 thereon for delivering a medium, such as a gaseous medium, to gas passage 34. A release and discharge of medium from canister 28 is effected through the depression of a release actuator 112 which in turn opens a seal 114 thus providing access for the product inside the canister 28, to escape therefrom, such as in the form of a gaseous medium. Although seal 114 is shown in schematic form only, it will be readily recognizable to one skilled in the art that seal 114 may take the form of any of the well-known spring-operated or biased seals adapted to be actuated for allowing access for a product inside a canister to escape therefrom in the form of a medium such as a gaseous medium. Depression of actuator 112 allows the product inside canister 28 to travel upward through a narrow access tube 116, through the open seal, and into nozzle 110 to escape therefrom. The shown mechanism is constructed such that the flow rate of the medium out of canister 28 is proportional to the amount of depression of actuator 112. Although shown in schematic form, the nozzle may be configured in any of the various conventional ways for expelling a medium therefrom. Canister 28 is sealed via a rigid cover 118 provided thereon. Cover 118 defines a threaded bore therein 120 which serves as a seat for and is complimentary to threaded bolt/screw 122 received therein. The threaded screw 122 is located inside a hole provided on actuator 112 such that it cooperates with therewith for actuating the same. The actuator 112/bore 120/screw 122 combination together form a system for a sustained delivery of predetermined flow rates of a medium according to which a threading-in of the screw moves the actuator 112 into its actuation mode, thus releasing the medium from canister 28. Various degrees of threading-in of screw 122 would effect correspondingly increasing flow rates of medium from canister 28. The shown configuration allows for a setting of the screw at a given threaded-in position for effecting a sustained release of medium from canister 28. Conversely, when it is desired to decrease the flow rate of the medium from canister 28, screw 122 is correspondingly threaded-out to a desired degree in order to effect the desired decrease. The relationship between a degree of threading-in or of threading-out of the screw versus flow rate of medium may easily be determined experimentally and indicated anywhere on the system or on an instruction manual accompanying the same.

Advantageously, the system for a sustained delivery as described above may be an inflation system for the sustained delivery of a predetermined flow rate of gaseous medium. Advantageously, such a system may be coupled to inlet 34 according to embodiments of the present invention described above for allowing a proportionally sustained delivery of predetermined amounts of intravenous fluid from the intravenous delivery system described in U.S. Pat. No. 5,505,708 mentioned above to a patient regardless of a spatial orientation of the intravenous delivery system. The use of the system for the sustained delivery of predetermined flow rates of a gaseous medium according to embodiments of the present invention in a system including a fluid discharge device, such as in systems shown in either of FIGS. 3 and 13, would further advantageously allow delivery of known flow rates of fluid from the shown intravenous delivery systems even in the presence of shifting forces. The system for the sustained delivery of predetermined flow rates of a medium according to embodiments of the present invention is not limited in its use to the intravenous delivery system described above, but may be used under any circumstances where sustained delivery of predetermined flow rates of a medium would be desirable, as readily within the knowledge of one skilled in the art.

Referring now to FIGS. 4-7 and 14, the operation of a preferred embodiment of the present invention when the container is an intravenous delivery bag is described. As seen in FIGS. 4-7 and 14, a preferred embodiment of the present invention includes a container of fluid which is a double-walled intravenous delivery bag as described above, and an outlet means of an intravenous delivery system including the bag which also includes the fluid discharge tube 10 as seen in FIGS. 4-7, or the channeled fluid guide as seen in FIG. 14. Although the operation of an embodiment of the present invention including a fluid delivery system comprising an intravenous delivery bag and the fluid discharge tube 10 will hereinafter be described as seen in FIGS. 4-7, it is to be understood that the general principle is substantially the same when applied to a system including the channeled fluid guide 100 instead, such as the one shown in FIG. 14, except that, in the latter case, fluid would flow along channels rather than through perforations and down a tube.

In operation, as the inflation system 4 is actuated, a gaseous medium enters the outer pressure envelope 22, as a result of which product storage envelope 24 is compressed uniformly, as shown for example in FIG. 5. As fluid drains from the product storage envelope 24 through the perforations of fluid discharge tube 10 as shown by the arrows entering perforations 16 of tube 10, the product storage envelope 24 becomes squeezed flat, and, where the fluid discharge tube 10 is present, encapsulates the same. Because of the sealing of the outer pressure envelopes with the product storage envelope at the sides thereof, as the product storage envelope collapses, it remains centered within the outer product envelope. In addition, advantageously, the domed end 18 of fluid discharge tube 10, allows a smooth contouring of the tube 20 by the product storage envelope 24 as it collapses, in this manner preventing any snagging of the product storage envelope by the end of the fluid discharge tube 10. The gas flowing into the outer pressure envelope creates a travelling pressure wave 26 (as shown by the hatchings in FIGS. 5-7) that moves down the product envelope 24 until the product envelope is completely drained.

FIG. 14 shows an intermediate stage of discharging fluid from a fluid delivery system comprising a channeled fluid guide 100, and is otherwise comparable to FIG. 6 described above.

Advantageously, embodiments of the invention as described by way of example with respect to FIGS. 3-7, 13 and 14 allow a draining of an intravenous delivery bag regardless of a spatial orientation of the same, and further provide an enhanced drainage of the bag by virtue of allowing enhanced access to different locations within the bag through a fluid discharge device as described above. The enhanced access is made possible by configuring the fluid discharge device to have a fluid intake portion that defines either perforations at at least end regions thereof, or channels along a length thereof, and that extends within the intravenous delivery bag. Such a configuration of a fluid discharge device ensures reliable delivery of fluid from an intravenous delivery bag even in the presence of shifting forces affecting the bag.

As noted previously, embodiments of a fluid discharge device according to the present invention are not limited to those shown with respect to FIGS. 1-7, 10, 11, 13 and 14. Other configurations are possible which would enhance a discharge of fluid from a container, such as when shifting forces are present. Examples of such other embodiments are provided in FIGS. 9a and 9b on the one hand, or in FIGS. 12a and 12b on the other hand.

Referring first to FIGS. 9a-9b, the fluid discharge device could include a network of tubes shaped either as a branching network 52 as shown in FIG. 9a, or a star-shaped network 54 as shown in FIG. 9b. Each of the networks 52 and 54 has a fluid intake portion 12 and a fluid outlet portion 14. As previously noted, as with any configuration of the fluid discharge tube according to the present invention, it is not necessary that the perforations extend along an entire length of the same as shown, as long as perforations are present at end regions of the fluid discharge device. In the case of the embodiments of FIGS. 9a and 9b, those end regions would correspond to the fluid outlet portion 14 as one end region, and to the tips of each of the branches of the network as another end region.

Referring first to FIGS. 12a-12b, the fluid discharge device could include a network of channeled fluid guides shaped either as a branching network 52 as shown in FIG. 12a, or a star-shaped network 54 as shown in FIG. 12b. Each of the networks 52 and 54 has a fluid intake portion 12 and a fluid outlet portion 14.

FIGS. 9a, 9b, 12a and 12b merely show examples for possible configurations of the fluid discharge device according to the present invention. Other configurations for the fluid discharge device according to the present invention would be within the knowledge of one skilled in the art. In particular, it would also be possible to have a branching network of tubes or channeled guides extending in three dimensions, or a star-shaped network extending in three dimensions, depending on the shape of the container of fluid and the particular circumstances under which the fluid discharge device would be used, as readily recognizable by one skilled in the art.

The present disclosure should not be construed in any limited sense other than that limited by the scope of the claims, having regard to the teachings herein and the prior art being apparent with the preferred form of the invention disclosed herein, and which reveals details of the structure of a preferred form necessary for a better understanding of the invention, and may be subject to change by the skilled person within the scope of the invention without departing from the concept thereof.

1. A fluid discharge device adapted to be coupled to a container for delivering fluid from the container, the fluid discharge device defining a fluid passageway and having a fluid intake portion and a fluid outlet portion, wherein:
the fluid intake portion is adapted to be placed within the container when the fluid discharge device is coupled to the container, and defines either perforations at least at end regions thereof, or channels extending along a length thereof, for discharging fluid from the container either through the perforations or along the grooves; and
the fluid outlet portion is adapted to deliver fluid discharged from the fluid intake portion to a region outside of the fluid intake portion.

2. The fluid discharge device of claim 1, wherein the fluid outlet portion is adapted to extend from the fluid intake portion outside the container when the fluid discharge device is coupled to the container for delivering fluid discharged from the fluid intake portion to a region outside of the container.

3. The fluid discharge device of claim 1, wherein the fluid outlet portion corresponds to an exit end of the fluid intake portion, and is adapted to be attached in an interior region of the container to an exit port for delivering fluid discharged from the fluid intake portion to a region outside of the container.

4. The fluid discharge device of claim 1, wherein the fluid discharge device comprises a single fluid discharge tube

5. The fluid discharge device of claim 1, wherein the fluid discharge device comprises a branching network of fluid discharge tubes

6. The fluid discharge device of claim 1, wherein the fluid discharge device comprises a star-shaped of fluid discharge tubes

7. The fluid discharge device of claim 1, wherein the fluid intake portion defines perforations extending along an entire length thereof.

8. The fluid discharge device of claim 7, wherein the perforations are distributed along the fluid discharge device in a staggered manner or in a uniform manner.

9. The fluid discharge device of claim 1, wherein the fluid intake portion defines perforations at least at lateral regions thereof.

10. The fluid discharge device of claim 1, wherein the perforations are cylindrical

11. The fluid discharge device of claim 1, wherein the fluid discharge device comprises a single channeled fluid guide.

12. The fluid discharge device of claim 1, wherein the fluid discharge device comprises a branching network of channeled fluid guides.

13. The fluid discharge device of claim 1, wherein the fluid discharge device comprises a star-shaped network of channeled fluid guides.

14. The fluid discharge device of claim 1, wherein the fluid discharge device has a cross-sectional profile in a plane perpendicular to its longitudinal axis which is one of X-shaped, star-shaped, Y-shaped or H-shaped.

15. The fluid discharge device of claim 1, wherein the fluid discharge device is made of a flexible material.

16. The fluid discharge device of claim 1, wherein the fluid discharge device is made of plastic.

17. The fluid discharge device of claim 1, wherein the length of the fluid intake portion is such that, when disposed inside the container, the fluid intake portion extends substantially along a length of an interior portion of the container.

18. The fluid discharge device of claim 1, wherein the fluid intake portion has a domed end.

19. The fluid discharge device of claim 1, wherein the container is an intravenous fluid storage and delivery container, and wherein the fluid discharge device is configured and sized to be coupled to the container for delivering fluid therefrom.

20. A combination comprising a container of fluid discharge device of claim 1, wherein the fluid discharge device is coupled to the container for delivering fluid therefrom.

21. The combination of claim 20, wherein the container is an intravenous fluid storage and delivery container.

22. The combination of claim 21, wherein the combination is an intravenous fluid storage and delivery system comprising:
a) a product storage envelope being generally rectangular and having a top, a bottom, a front wall, a back wall and two oppositely disposed sides, the product storage envelope being adapted to store a fluid therein;
b) an outer pressure envelope, also being generally rectangular and having a top, a bottom, a front wall, a back wall, and two oppositely disposed sides, said outer pressure vessel being placed so as to completely surround and cover said product storage envelope and further such that said bottom and sides of said outer pressure envelope are in continuous sealing contact with the bottom and sides of said product envelope such that a common seam exists around the perimeter of the envelopes;
c) outlet means in communication with said product storage envelope to enable extraction of the fluid from said product storage envelope, the outlet means including the fluid discharge device;
d) inlet means in communication with said outer pressure envelope to permit entry of a flow of external fluid into said outer pressure envelope.

23. The combination of claim 22, wherein the external fluid is a gaseous medium, the combination further comprising:
(a) a canister of gas removably connected to said inlet means; and
(b) means to control the flow of said gas into said inlet means.

24. The combination of claim 22, further comprising means for controlling the flow of external fluid into said inlet means such that the flow creates a traveling pressure wave within said outer pressure envelope.

25. The combination of claim 24, wherein said traveling pressure wave is adapted to be delivered with equal pressure to both a front wall and a back wall of said product storage envelope.

26. The combination of claim 23, wherein the means to control include valve means.

27. The combination of claim 23, wherein the means to control include a system for a sustained delivery of predetermined flow rates of gaseous medium to the inlet means.

28. The combination of claim 27, wherein the system for a sustained delivery includes:
an actuator adapted to be depressed for effecting a release of gaseous medium from the canister, and configured such that amounts of depression of the actuator are proportional to corresponding flow rates of gaseous medium being delivered from the canister; and
a mechanism coupled to the actuator for effecting various degrees of depression of the actuator and for sustaining the actuator at each of the various degrees of depression for causing a proportional sustained delivery of predetermined flow rates of the gaseous medium from the canister.

29. The combination of claim 28, wherein the mechanism includes:
a rigid cap disposed on the canister and defining a threaded bore therein; and
a correspondingly threaded screw threaded into the threaded bore and coupled to the actuator such that various degrees of threading-in or of threading-out of the screw cause corresponding various degrees of depression of the actuator and sustain the actuator at each of the various degrees of depression.

30. The combination of claim 21, wherein the combination is an intravenous fluid storage and delivery system comprising:
a) a product storage envelope being generally rectangular and having a top, a bottom, a front wall, a back wall and two oppositely disposed sides, the product storage envelope being adapted to store a fluid therein;
b) an outer pressure envelope, also being generally rectangular and having a top, a bottom, a front wall, a back wall, and two oppositely disposed sides, said outer pressure vessel being placed so as to completely surround and cover said product storage envelope and further such that said bottom and sides of said outer pressure envelope are in continuous sealing contact with the bottom and sides of said product envelope such that a common seam exists around the perimeter of the envelope and further such that the top of said outer pressure envelope is attached to said top of said inner product envelope with non-continuous seams and with a spatial gap therebetween, said non-continuous seams permitting the flow of gas or liquid from said spatial gap into said outer pressure envelope;
c) outlet means in communication with said product storage envelope to enable extraction of the fluid from said product storage envelope, the outlet means including the fluid discharge device;
d) inlet means in communication with said outer pressure envelope to permit entry of a flow of gas into said outer pressure envelope through said spatial gap;
e) a canister of gas removably connected to said inlet means; and
f) means to control the flow of the gas from the canister into said inlet means.

31. The combination of claim 26, wherein the means to control is a valve means for controlling the flow of the gas into said inlet means such that the flow of the gas into said outer pressure envelope creates a traveling pressure wave within said outer pressure envelope.

32. The combination of claim 31, wherein said traveling pressure wave is delivered with equal pressure to both a front wall and a back wall of said inner product envelope.

33. The combination of claim 30, wherein the means to control include a system for a sustained delivery of predetermined flow rates of gaseous medium to the inlet means.

34. The combination of claim 33, wherein the system for a sustained delivery includes:
an actuator adapted to be depressed for effecting a release of gaseous medium from the canister, and configured such that amounts of depression of the actuator are proportional to corresponding flow rates of gaseous medium being delivered from the canister; and
a mechanism coupled to the actuator for effecting various degrees of depression of the actuator and for sustaining the actuator at each of the various degrees of depression for causing a proportional sustained delivery of predetermined flow rates of the gaseous medium from the canister.

35. The combination of claim 34, wherein the mechanism includes:
a rigid cap disposed on the canister and defining a threaded bore therein; and
a correspondingly threaded screw threaded into the threaded bore and coupled to the actuator such that various degrees of threading-in or of threading-out of the screw cause corresponding various degrees of depression of the actuator and sustain the actuator at each of the various degrees of depression.

36. A method of making the intravenous fluid storage and delivery system of claim 30, comprising:
a) folding a first, generally rectangular sheet of plastic;
b) folding a second generally rectangular sheet of plastic, said second sheet being somewhat longer than said first sheet;
c) placing said second sheet over said first sheet, thereby creating a double walled structure wherein said first, generally rectangular sheet of plastic forms an inner product envelope; and the second generally rectangular sheet of plastic forms an outer pressure envelope, said placement also forming a spatial gap between the fold of the first sheet and the fold of the second sheet;
d) sealing the open seams between them continuously around their perimeters;
e) installing the fluid discharge device into said seams in communication with said first sheet;
f) tack seaming the top portion of the second sheet to the top of the first sheet; and
g) installing an inlet port in said spatial gap.

37. The method of claim 36 further comprising attaching a means for inflating said outer pressure envelope to said inlet port.

38. The method claim 36, further comprising the step of filling the inner product envelope with a fluid.

39. A system for a sustained delivery of predetermined flow rates of a medium comprising:
an actuator adapted to be depressed for effecting a release of gaseous medium from a canister, and configured such that amounts of depression of the actuator are proportional to corresponding flow rates of medium being delivered from the canister; and
a mechanism coupled to the actuator for effecting various degrees of depression of the actuator and for sustaining the actuator at each of the various degrees of depression for causing a proportional sustained delivery of predetermined flow rates of the medium from the canister.

40. The combination of claim 39, wherein the mechanism includes:
a rigid cap disposed on the canister and defining a threaded bore therein; and
a correspondingly threaded screw threaded into the threaded bore and coupled to the actuator such that various degrees of threading-in or of threading-out of the screw cause corresponding various degrees of depression of the actuator and sustain the actuator at each of the various degrees of depression.

41. A combination comprising the system of claim 28 coupled to an intravenous fluid storage and delivery system comprising:
g) a product storage envelope being generally rectangular and having a top, a bottom, a front wall, a back wall and two oppositely disposed sides, the product storage envelope being adapted to store a fluid therein;
h) an outer pressure envelope, also being generally rectangular and having a top, a bottom, a front wall, a back wall, and two oppositely disposed sides, said outer pressure vessel being placed so as to completely surround and cover said product storage envelope and further such that said bottom and sides of said outer pressure envelope are in continuous sealing contact with the bottom and sides of said product envelope such that a common seam exists around the perimeter of the envelope and further such that the top of said outer pressure envelope is attached to said top of said inner product envelope with non-continuous seams and with a spatial gap therebetween, said non-continuous seams permitting the flow of gas or liquid from said spatial gap into said outer pressure envelope;
i) outlet means in communication with said product storage envelope to enable extraction of the fluid from said product storage envelope, the outlet means including the fluid discharge device;
j) inlet means in communication with said outer pressure envelope to permit entry of a flow of gas into said outer pressure envelope through said spatial gap;
k) a canister of gas removably connected to said inlet means, the system for a sustained delivery being coupled to the canister from delivering gas therefrom
